(19) United States
(12) Patent Application Publication
(10) Pub. No.: US 2004/0152212 A1
(43) Pub. Date: Aug. 5, 2004

(54) Optical detection method for protein microarray

(75) Inventors: Yi-You Huang, Taipei (TW); Hsin-Yun Hsu, Taipei (TW)

Correspondence Address: Haverstock & Owens LLP, 162 North Wolfe Road, Sunnyvale, CA, 94086, US

(73) Assignee: National Taiwan University

(21) Appl. No.: 10/758,303

(22) Filed: Jan. 14, 2004

(30) Foreign Application Priority Data
Jan. 15, 2003 (TW) 092100821

(51) Int. Cl. G01N 33/543

(52) U.S. Cl. 436/518

(57) ABSTRACT

An optical detection method for a protein microarray is disclosed. The optical detection method for the protein microarray includes steps of providing a capture molecule, recognizing a biomolecule on the protein microarray via the capture molecule, providing a primer to connect with the capture molecule, amplifying a signal of the primer on the capture molecule via a rolling circle amplification system, and detecting the amplified signal via a nanoparticle probe.

### FIELD OF THE INVENTION

This invention relates to an optical detection method for a protein microarray, and more particularly to an optical detection method for a protein microarray using a nanoparticle probe combined with a rolling circle amplification system.

### BACKGROUND OF THE INVENTION

Since the research of DNA microarray has made a breakthrough, many study groups make every endeavor to develop protein microarray chip. The detector for the protein microarray chip which has good performance and high efficacy is also developed devotedly by the research fellows. At present, in the field of medical research, the immunoassay is the most popular and powerful analysis technique. The specificity of analysis comes from the great recognition between the antibody and the corresponding antigen. In most immunoassays, the key point is the solid phase substrate, which is designed for the convenience of washing and isolating, and can be used for immobilizing the antibody or antigen thereon. Another key point of the immunoassay is the detection of the combination of particular molecules. A common detection method is using labeled molecule, such as radioisotope, fluorescence and enzyme.

Until now, one of the disadvantages of all detection kits is the complicated process and the loss of sensitivity. The problems of the radioisotope are the safety issue and the dispute for waste treatment. Therefore, the fluorescence label rather than the radioisotope label is used by most researcher in the application of the standard microarray. Although the detection of fluorescence needs highly precise and expensive fluorescence microscope and scanner, and is strongly influenced by the environmental factors, there is still no other new method or standard for reading the detection signal which can replace the detection of fluorescence. However, the low intensity of the fluorescence is a big challenge for quantitative analysis. In addition, the low stability and the bleaching problem of the fluorescence also need to be overcome.

Therefore, the present invention provides a new optical detection method, which uses a nanoparticle probe to detect the signal of the protein microarray chip.

### SUMMARY OF THE INVENTION

It is an object of the present invention to provide an optical detection method for a protein microarray using a nanoparticle probe combined with a rolling circle amplification system to increase the sensitivity of the optical detection.

In accordance with an aspect of the present invention, the optical detection method for a protein microarray includes steps of providing a capture molecule, recognizing a biomolecule on the protein microarray via the capture molecule, providing a primer to connect with the capture molecule, amplifying a signal of the primer on the capture molecule via a rolling circle amplification system, and detecting the amplified signal via a nanoparticle probe.

Preferably, the capture molecule is one selected from a group consisting of an antibody, a biomarker, a protein receptor, a carbohydrate and a peptide.

Preferably, the biomolecule is one selected from a group consisting of an antigen, a ligand, a protein, a carbohydrate and a peptide.

Preferably, the primer is a single-strand oligonucleotide of 20-80 bp.

Preferably, the 5′ end of the primer is modified with an amino group to connect with the capture molecule.

Preferably, the rolling circle amplification system comprises a DNA polymerase, a circular template, nucleotides (dNTP) and a buffer system.

Preferably, the circular template has a sequence complementary to the primer to hybridize with the primer.

Preferably, the rolling circle amplification system generates a single-strand DNA molecule connected with the primer and having tandemly repeats of a sequence complementary to the circular template via the DNA polymerase.

Preferably, the circular template has a nucleotide sequence of 25-100 bp.

Preferably, the nanoparticle probe is a nanoparticle modified with a single-strand oligonucleotide.

Preferably, the nanoparticle is one of a nanogold and a quantum dot.

Preferably, a length of the single-strand oligonucleotide is 10-60 bp.

Preferably, the 5′ end of the single-strand oligonucleotide is modified with an —SH group to react strongly with the surface of the nanoparticle.

Preferably, the nanoparticle is a sphere or a polyhedron.

In accordance with another aspect of the present invention, the optical detection system for a protein microarray includes a capture molecule for recognizing a biomolecule on the protein microarray, a primer for connecting with the capture molecule, a rolling circle amplification system for amplifying a signal of the primer on the capture molecule, and a nanoparticle probe for detecting the amplified signal.

Preferably, the primer is a single-strand oligonucleotide of 20-80 bp.

Preferably, the 5′ end of the primer is modified with an amino group to connect with the capture molecule.

Preferably, the rolling circle amplification system comprises a DNA polymerase, a circular template, nucleotides (dNTP) and a buffer system.

Preferably, the circular template has a nucleotide sequence of 25-100 bp.

Preferably, the nanoparticle probe is a nanoparticle modified with a single-strand oligonucleotide.

Preferably, the nanoparticle is one of a nanogold and a quantum dot.

Preferably, a length of the single-strand oligonucleotide is 10-60 bp.

The above objects and advantages of the present invention will become more readily apparent to those ordinarily skilled in the art after reviewing the following detailed description and accompanying drawings, in which:

### BRIEF DESCRIPTION OF THE DRAWINGS

FIG. 1 is a schematic view showing the optical detection method for a protein microarray according to a preferred embodiment of the present invention;

FIG. 2 shows the TEM view of the nanogold particles according to a preferred embodiment of the present invention;

FIG. 3 shows a spectrum analysis chart of the oligonucleotide modified nanogold particles according to a preferred embodiment of the present invention;

FIG. 4 shows the TEM view of the nanogold particles after signal amplification according to a preferred embodiment of the present invention;

FIG. 5 shows an electrophoresis diagram for the product of RCA according to a preferred embodiment of the present invention; and

FIG. 6 shows a plot of DNA concentration versus absorption when using the nanogold particles to detect the DNA molecules according to a preferred embodiment of the present invention.

### DETAILED DESCRIPTION OF THE PREFERRED EMBODIMENT

The great function of the present invention comes from the high specificity between the antibody and the particular antigen epitope. Many important biomarkers of cancers, infectious diseases, or biochemical reactions have very low concentrations in blood, body fluids or tissues, so that they are hard to be detected by conventional immunoassays. Especially for those samples with little and limited amounts or antigens with extremely low concentrations, higher sensitivity and specificity are required. The present invention uses the new rolling circle amplification (RCA) technique accompanying with the nanoparticle technique for detecting the protein microarray to increase the sensitivity of the immunoassay.

The rolling circle amplification system of the present invention uses DNA polymerase for driving the signal amplification. The DNA polymerase can replicate the circular nucleotide template in a linear or geometric kinetics manner under isothermal conditions. Using single or plural primers, the rolling circle amplification system can generate hundreds of tandemly repeats of a sequence in a few minutes. Thus, the rolling circle amplification can be used to amplify the signal of an antigen-antibody immune reaction. The 5′ end of the primer is connected to the antibody. In the presence of a circular template, a DNA polymerase, nucleotides (dNTP) and a buffer system, the rolling circle amplification system can generate an amplified single-strand DNA molecule connected with the primer and having tandemly repeats of a sequence complementary to the circular template. The amplified DNA molecule can be detected by many methods, such as adding haptens, using fluorescence-labeled nucleotides, or hybridizing by complementary oligonucleotide probe labeled with fluorescence or enzyme. In the present invention, a nanoparticle modified with oligonucleotide is used as a detection probe, in which the nanoparticle can be a nanogold or a quantum dot. Therefore, the detection technique of the present invention is a new method for amplifying the interaction signal of molecular recognition (such as antigen-antibody immune reaction).

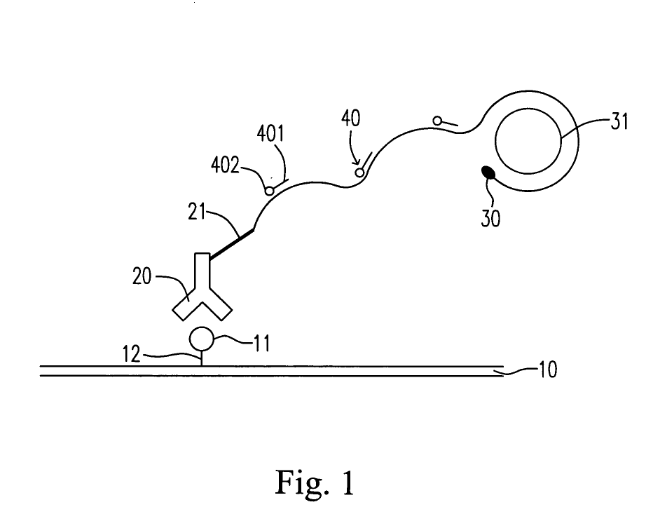

Please refer to FIG. 1 showing the optical detection method for a protein microarray of the present invention. A capture molecule 20 is provided for recognizing a biomolecule 11 on the protein microarray 10. The capture molecule 20 can be an antibody, a biomarker (such as a tumor marker), a protein receptor, a carbohydrate or a peptide. The biomolecule 11 can be an antigen, a ligand, a protein, a carbohydrate or a peptide. Then a primer 21 is provided, in which the 5′ end of the primer 21 is modified with an amino group, so that the primer 21 is easy to connect with the capture molecule 20. The primer is a single-strand oligonucleotide of 20-80 bp.

Subsequently, a rolling circle amplification system is used to amplify the signal of the primer 21 on the capture molecule 20. The rolling circle amplification system includes a DNA polymerase 30, a circular template 31, nucleotides (dNTP) and a buffer system. The nucleotide sequence length of the circular template 31 is 25-100 bp, preferably 30-50 bp, and the circular template 31 has a sequence complementary to the primer 21 so as to hybridize with the primer 21. Via the DNA polymerase 30, a single-strand DNA molecule connected with the primer 21 and having tandemly repeats of a sequence complementary to the circular template 31 is generated.

Therefore, the reaction signal of the capture molecule 20 and the biomolecule 11 on the protein microarray 10 can be amplified through the amplified DNA, and the amplified DNA is detected by a nanoparticle probe 40. The nanoparticle probe 40 is a nanoparticle 402 modified with a single-strand oligonucleotide 401, in which the nanoparticle 402 can be a nanogold or a quantum dot. The nanoparticle 402 is a sphere or a polyhedron, and the length of the single-strand oligonucleotide 401 is 10-60 bp, preferably 15-25 bp. In addition, the 5′ end of the single-strand oligonucleotide 401 is modified with an —SH group to react strongly with the surface of the nanoparticle 402. Since the single-strand oligonucleotide 401 has a sequence complementary to the amplified DNA, the reaction signal of the protein microarray 10 can be further detected by the hybridization of the complementary sequences.

Moreover, the hybridization signal of the nanoparticle probe with the complementary target sequence is visible light, so it can be easily detected without using precise and expensive fluorescence instruments. The sensitivity can be further increased by silver enhancement technique, which reduces silver ion to silver metal via hydroquinone, so that the signal of the protein microarray can be detected by a conventional flatbed scanner. Therefore, the present invention provides an optical detection method using oligonucleotide modified nanoparticle as a probe to detect the interaction of particular protein molecules on the surface of miniaturized chip. The present invention combines the chip immobilization technique and the signal amplification system to enable high throughput parallel detections.

The detection principle of the nanoparticle probe in the present invention relies on the surface plasmon resonance (SPR), so that the use of fluorescence and radioisotope can be avoided. Since high compatibility exists between the nanoparticle and the biomolecule, the stability of the sample can be highly increased. Compared to the detection of fluorescence, the fluorescence has problems of low sensitivity and bleaching, and the detection of fluorescence is limited to a short period, so it must be detected as soon as possible. However, the detection of the nanoparticle has no such problems. Via the steps of filtration and dialysis, the nanoparticle can be stored at 4° C. for several months. The increase of the stability makes long time or repeated sample analysis possible. In addition, the influence of the chemical and physical environment to the strength of the signal is highly reduced, so the reproducibility of the experiment results can be intensified. Different analyses and chip detections provide higher comparability therebetween, and also provide applicable potential for miniaturization process. Compared to the microarray chip monitored by fluorescence, the high signal-to-noise ratio provided by the nanagold particle enables the size limitation of the distribution points down to sub-micrometer. For parallel analysis, one significant advantage of the present invention is that the arrangement density of the distribution points for the microarray can be increased several order of magnitude. Therefore, the nanoparticle is suitable for detecting the interaction of particular biomolecules on the microarray chip. According to the method of the present invention, fluorescence instruments are no longer needed for reading the optical detection, and thus, a new field and direction of the chip detection system is provided.

The features and advantages of the present invention are illustrated with the following embodiments. The particular materials and the amounts thereof in the following embodiments are used as examples but not to limit the present invention.

Embodiment 1: Preparation of Nanogold Particles with Diameter of 16 nm by Citrate Reduction of HAuCl₄

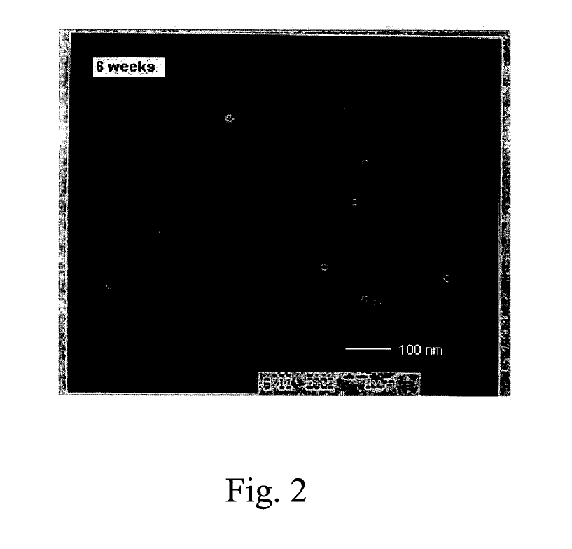

All glass containers are immersed in aqua regia (3 parts HCl+1 part HNO₃), and then washed with ddH₂O and baked before they are used. A HAuCl₄ solution (0.01%, 50 mL) is prepared and then heated and refluxed till boiling. A trisodium citrate solution (1%, 1 mL) used as a reducing agent is immediately added therein to reduce the gold ion to gold metal. The color of the solution would change from light yellow to dark red. After the color change, the solution is refluxed continuously for 15 minutes and then cooled down on ice. The solution is filtered through 0.22 mm nylon filter and dialyzed to remove the impurities, such as the salts. Then the solution is stored at 4° C. The TEM (Transmission Electron Microscope) view of the produced nanogold particles is shown in FIG. 2.

The preparation of the nanogold particles with different particle sizes is similar to the above method, only the kind and the amount of the reducing agent are adjusted according to the desired particle size. For example, tannic acid can be used as a reducing agent to prepare the nanogold particle with the particle size less than 5 nm. The nanogold particle size suitable for the present invention is between 1 nm to 100 nm, preferably 5 nm to 30 nm.

Embodiment 2: Preparation of Oligonucleotide Modified Nanogold Particles

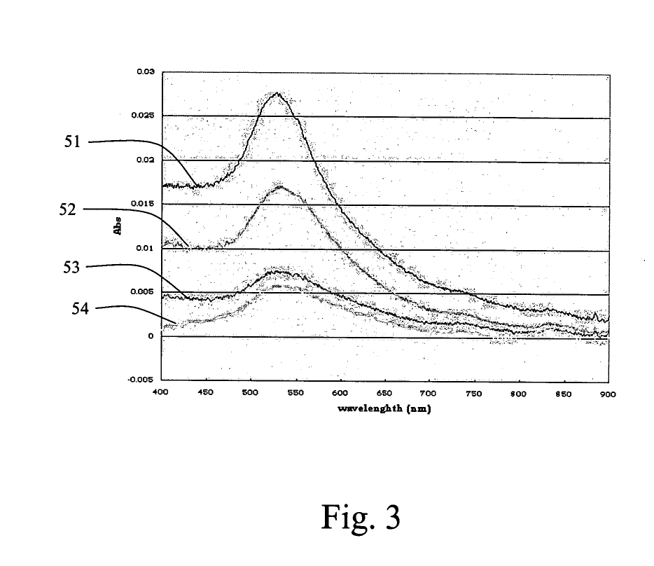

5 mL nanogold particle solution is mixed with prepared oligonucleotide (about 3.1 mM, equal to the concentration of 1× probe in FIG. 3) and stand for 16 hours. Then the solution is added in 0.1 M NaCl and 10 mM phosphate buffer (pH 7) and stand for 40 hours. Subsequently, the solution is centrifugated at 14,000 rpm for 25 minutes to obtain a dark red oily pellet and remove the supernatant. The pellet is washed with 5 mL, 0.1 M NaCl and 10 mM phosphate buffer (pH 7), and then centrifugated to remove the supernatant. The pellet is resuspended in 5 mL, 0.3 M NaCl/10 mM phosphate buffer (pH 7)/0.01% azide solution to result in an evenly mixed nanogold probe solution.

FIG. 3 shows a spectrum analysis chart of the oligonucleotide modified nanogold particles. The absorption spectrums 51, 52, 53 and 54 of the nanogold particles respectively modified with 0, ½, 1 and 2× probe (1× probe represents 3.1 mM oligonucleotide) are shown in FIG. 3. The surface modification degree of the nanogold particles can be calculated according to the reduction of the absorption peak at 520 nm.

In addition, the oligonucleotide modified nanogold particles of the present invention can be recovered.

Embodiment 3: Rolling Circle Amplification (RCA) System Accompanying with Nanogold Particles

The reaction solution (50 mL) of the rolling circle amplification system includes 0.05/0.06 nmole circular template/primer (the 5′ end thereof is modified with an amino group to easily connect with the capture molecule, such as the antibody), 1 mM dNTP, 1× Reaction Buffer (10 mM Tris-HCl (pH 7.5), 5 mM MgCl₂, 7.5 mM dithiothreitol), DNA polymerase (such as E. coli DNA Polymerase I, ˜5 units), and ddH₂O which is added until the total volume is 50 mL. The reaction solution is water bathed at 37° C. for 1 hour.

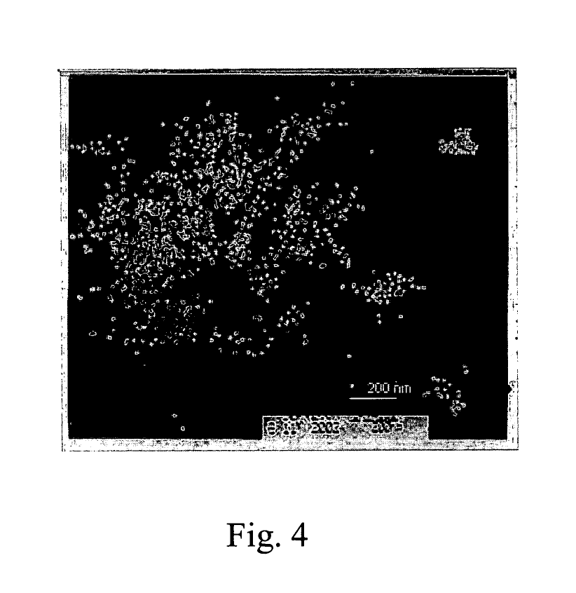

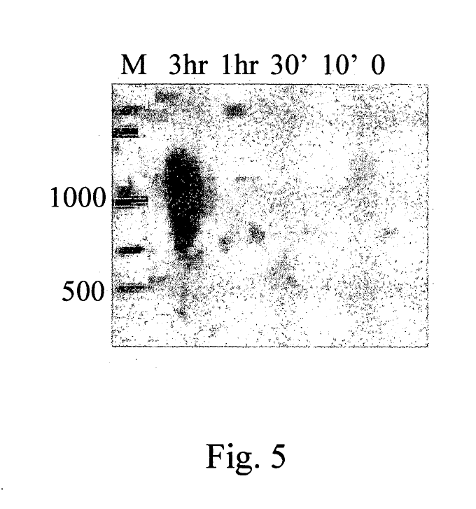

The TEM view of the nanogold particles after signal amplification is shown in FIG. 4. FIG. 5 is an electrophoresis diagram to confirm the product of RCA.

Embodiment 4: Optical Detection of Nanogold Particles

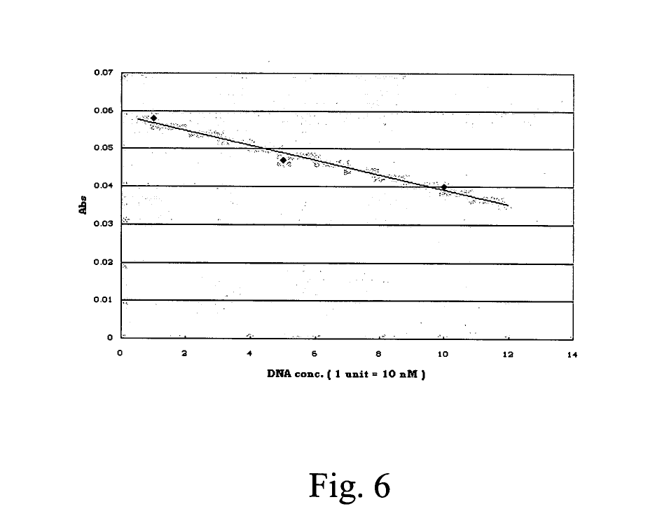

The biomolecule, such as DNA or protein, is immobilized on the solid phase substrate via a C3˜C12 linker, which is modified with an —SH group at one end and with a —COOH group at the other end, and via SMPB or EDC/NHS as a cross-link reagent. After rolling circle amplification, the amplified signal is detected by the oligonucleotide modified nanogold particles. Then the enhanced scattering effect generated by the nanogold particles can be detected via the surface plasmon resonance (SPR). In the present invention, the absorption of the nanogold particles at the wavelength around 520 nm of the characteristic peak in the spectrum is detected by an ELISA reader. The more target molecules, the more nanogold particles are combined thereon, which results in the enhancement of the scattering effect and the reduction of the absorption. FIG. 6 shows a plot of DNA concentration versus absorption when using the nanogold particles to detect the DNA molecules.

In conclusion, the present invention provides an optical detection method for a protein microarray using the nanoparticle probe combined with the rolling circle amplification system, which has high sensitivity and can be detected by a conventional flatbed scanner.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An optical detection method for a protein microarray, comprising steps of:
providing a capture molecule;
recognizing a biomolecule on said protein microarray via said capture molecule;
providing a primer to connect with said capture molecule;
amplifying a signal of said primer on said capture molecule via a rolling circle amplification system; and
detecting said amplified signal via a nanoparticle probe.

2. The method according to claim 1 wherein said capture molecule is one selected from a group consisting of an antibody, a biomarker, a protein receptor, a carbohydrate and a peptide.

3. The method according to claim 1 wherein said biomolecule is one selected from a group consisting of an antigen, a ligand, a protein, a carbohydrate and a peptide.

4. The method according to claim 1 wherein said primer is a single-strand oligonucleotide of 20-80 bp.

5. The method according to claim 1 wherein the 5′ end of said primer is modified with an amino group to connect with said capture molecule.

6. The method according to claim 1 wherein said rolling circle amplification system comprises a DNA polymerase, a circular template, nucleotides (dNTP) and a buffer system.

7. The method according to claim 6 wherein said circular template has a sequence complementary to said primer to hybridize with said primer.

8. The method according to claim 7 wherein said rolling circle amplification system generates a single-strand DNA molecule connected with said primer and having tandemly repeats of a sequence complementary to said circular template via said DNA polymerase.

9. The method according to claim 6 wherein said circular template has a nucleotide sequence of 25-100 bp.

10. The method according to claim 1 wherein said nanoparticle probe is a nanoparticle modified with a single-strand oligonucleotide.

11. The method according to claim 10 wherein said nanoparticle is one of a nanogold and a quantum dot.

12. The method according to claim 10 wherein a length of said single-strand oligonucleotide is 10-60 bp.

13. The method according to claim 10 wherein the 5′ end of said single-strand oligonucleotide is modified with an —SH group to react strongly with the surface of said nanoparticle.

14. The method according to claim 10 wherein said nanoparticle is a sphere or a polyhedron.

15. An optical detection system for a protein microarray, comprising:
a capture molecule for recognizing a biomolecule on said protein microarray;
a primer for connecting with said capture molecule;
a rolling circle amplification system for amplifying a signal of said primer on said capture molecule; and
a nanoparticle probe for detecting said amplified signal.

16. The method according to claim 15 wherein said primer is a single-strand oligonucleotide of 20-80 bp.

17. The system according to claim 15 wherein the 5′ end of said primer is modified with an amino group to connect with said capture molecule.

18. The system according to claim 15 wherein said rolling circle amplification system comprises a DNA polymerase, a circular template, nucleotides (dNTP) and a buffer system.

19. The system according to claim 18 wherein said circular template has a nucleotide sequence of 25-100 bp.

20. The method according to claim 15 wherein said nanoparticle probe is a nanoparticle modified with a single-strand oligonucleotide.

21. The method according to claim 20 wherein said nanoparticle is one of a nanogold and a quantum dot.

22. The method according to claim 20 wherein a length of said single-strand oligonucleotide is 10-60 bp.
